(19) United States
(12) Patent Application Publication
(10) Pub. No.: US 2007/0260090 A1
(43) Pub. Date: Nov. 8, 2007

(54) Highly Steroselective Synthesis of Sertraline

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Jonnala Sambi Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/569,721

(22) PCT Filed: Jun. 3, 2005
(86) PCT No.: PCT/IN05/00182

§ 371 Date: Nov. 28, 2006

(51) Int. Cl. C07C 211/45

(52) U.S. Cl. 564/308

(57) ABSTRACT

The present invention relates to a process for highly stereoselective synthesis of sertraline and sertraline intermediate. Thus, the mixture of 4-(3,4-Dichlorophenyl)-3,4-dihydro-N-methyl-1(2H)-naphthalenimine, 5% Pd/CaCO₃, water and methanol is taken in a hydrogenation flask and then subjected to hydrogenation under a hydrogen pressure of 0.5 Kg at 20-35° C. for 3 hours 30 minutes. The catalyst is removed by filtration and the solvent is evaporated completely under vacuum to obtain cis-(±)-4-(3,4-dichlorophenyl)-1,2,3,4-tetrahydro-N-methyl-naphthalen amine. (trans-(±): 0.2).

### FIELD OF THE INVENTION

The present invention relates to a process for highly stereoselective synthesis of sertraline and sertraline intermediate.

### BACKGROUND OF THE INVENTION

U.S. Pat. No. 4,536,518, which is herein incorporated by reference, disclosed cis-isomeric derivatives, of 4-phenyl-1,2,3,4-tetrahydro-1-naphthalenamine and their pharmaceutically acceptable salts. These compounds act to block the synaptosomal uptake of serotonin (5-hydroxy-tryptamine), thereby alleviating serotonin abnormalities at central receptor sites. Among them sertraline, chemically (1S,4S)-4-(3,4-dichlorophenyl)-1,2,3,4-tetrahydro-N-methyl-1-naphthalenamine is a selective serotonin reuptake inhibitor (SSRI). Sertraline is represented by the following structure:

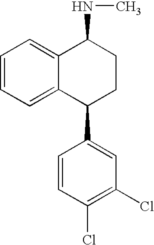

Processes for the preparations of sertraline, it's pharmaceutically acceptable salts and related compounds were described in U.S. Pat. No. 4,536,518, U.S. Pat. No. 6,552,227, PCT publication No. WO 99/57093 A1, U.S. Pat. No. 6,232,501, U.S. Pat. No. 6,034,274 and PCT publication No. WO 01/16089.

The key step in the preparation of sertraline or a salt thereof is the hydrogenation of the racemic 4-(3,4-dichlorophenyl)-3,4-dihydro-N-methyl-1(2H)-naphthalenimine of formula A: 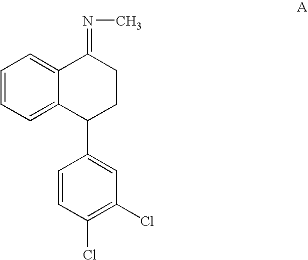
to obtain the isomers of formulae:

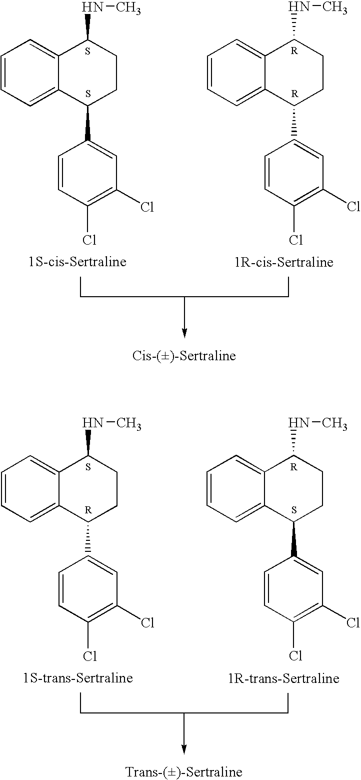

Of the four isomers formed, 1S-cis isomer is desired sertraline and so, all the other three isomers has to be separated from the mixture to obtain the desired isomer. Thus, the preparation of sertraline from racemic sertraline may be represented schematically as below:

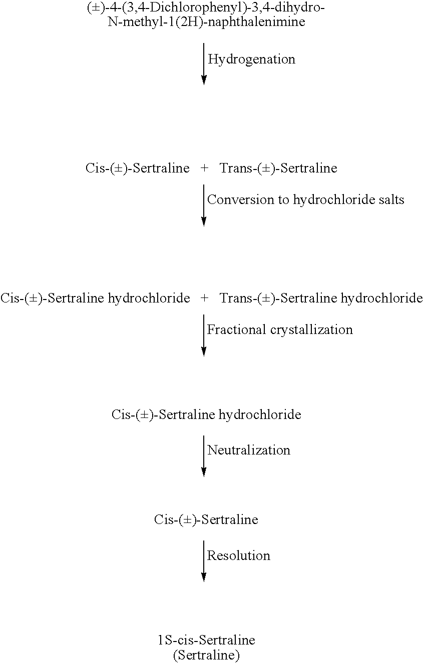

Sertraline hydrochloride may also be prepared from 4(S)-(3,4-dichlorophenyl)-3,4-dihydro-N-methyl-1(2H)-naphthalenimine of formula B:

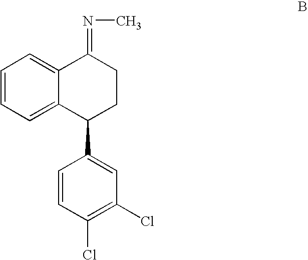

The preparation of sertraline from 4(S)-(3,4-dichlorophenyl)-3,4-dihydro-N-methyl-1(2H)-naphthalenimine may be represented schematically as below:

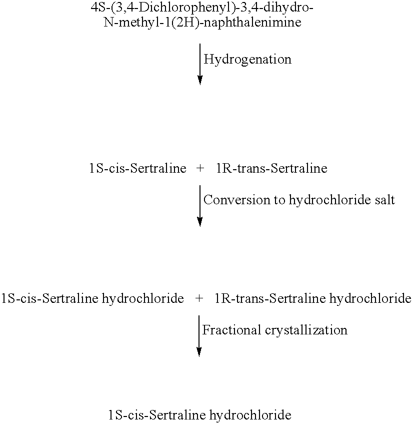

In the above processes, the key step is the hydrogenation step.

U.S. Pat. No. 4,536,518 described hydrogenation of imine compound of formula A using Pd/C at room temperature. In this step cis-(±), trans-(±)-sertralines are obtained in about 3:1 ratio.

U.S. Pat. No. 6,552,227 described hydrogenation of imine compound of formula A using Pd/C or PtO₂ at a temperature above 40° C. to obtain cis-(±) and trans-(±) sertralines in higher ratio of cis-(±)-sertraline over the trans-(±) sertraline compound than that obtained in U.S. Pat. No. 4,536,518. According to the processes described in the published patent application, the hydrogenation resulted in the formation of cis-(±) and trans-(±) sertralines in the ratio at the maximum of 12:1.

PCT publication No. WO 99/57093 A1 described hydrogenation of imine compound of formula A using Pd applied on a carrier pre-treated with an alkyl halide to obtain cis-(±) and trans-(±) isomers in the ratio of at the maximum of 19:1.

U.S. Pat. No. 6,232,501 describes hydrogenation of 4-(3,4-dichlorophenyl)-3,4-dihydro-N-methyl-1(2H)-naphthalenimine or 4-(3,4-dichloro phenyl)-3,4-dihydro-N-methyl-1(2H)-naphthalenimine-N-oxide to obtain cis-trans sertraline using copper containing catalyst. The hydrogenation is required to carried out at very high pressure (10-15 bars) and/or at high temperatures (100° C.-150° C.).

U.S. Pat. No. 6,034,274 describes hydrogenation of 4-(3,4-dichloro phenyl)-3,4-dihydro-N-methyl-1(2H)-naphthalenimine-N-oxide to obtain cis-trans sertraline, followed by conversion into hydrochloride salts and fractional crystallization of cis (±)-sertraline hydrochloride.

WO 01/16089 describes reductive amination of 4-(3,4-dichlorophenyl)-3,4-dihydro-1-(2H)-naphthalenone to obtain cis-trans sertraline, followed by conversion into hydrochloride salts and fractional crystallization of cis(±)-sertraline hydrochloride.

U.S. Pat. No. 6,506,940 described the conversion of one stereoisomer of sertraline into another stereoisomers via 4-(3,4-dichlorophenyl)-3,4-dihydro-N-methyl-1(2H)-naphthalenimine.

U.S. Pat. No. 6,723,878 described a method of hydrogenation of 4-(3,4-dichlorophenyl)-3,4-dihydro-N-methyl-1(2H)-naphthalenimine in the presence of a dehalogenating agent to obtain cis-trans sertraline. The process requires high pressures.

Deschloro impurities of Sertraline, namely cis-(±)-4-(3 or 4-chlorophenyl)-1,2,3,4-tetrahydro-N-methyl-naphthalenamine and cis-(±)-4-phenyl-1,2,3,4-tetrahydro-N-methyl-naphthalenamine are common impurities of sertraline and formed when hydrogenation is carried out at high temperature and/or high pressures.

WO 03/099761 describes reductive amination of 4-(3,4-dichlorophenyl)-3,4-dihydro-1-(2H)-naphthalenone with methylamine to obtain cis-trans sertraline, followed by conversion into hydrochloride salts and fractional crystallization of cis(±)-sertraline hydrochloride.

According to the present invention, it has been found that the imine compounds of above mentioned compounds can be hydrogenated in a highly stereoselective manner in a simple procedure to obtain the cis-isomers of sertraline.

### DETAILED DESCRIPTION OF THE INVENTION

According to one aspect of the present invention, there is provided a process for preparing cis-(±)-4-(3,4-dichlorophenyl)-1,2,3,4-tetrahydro-N-methyl-naphthalenamine of formula I: 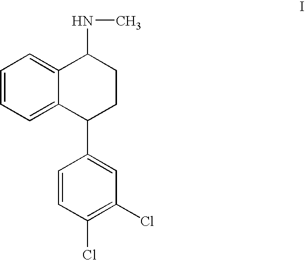
which comprises hydrogenating 4-(3,4-dichlorophenyl)-3,4-dihydro-N-methyl-1(2H)-naphthalenimine of formula II: 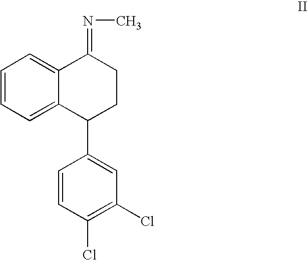
with palladium supported on an alkaline earth metal carbonate to obtain substantially pure cis-(±)-4-(3,4-dichlorophenyl)-1,2,3,4-tetrahydro-N-methyl-naphthalenamine of formula I.

The preferable alkaline earth metal carbonate is CaCO₃ or BaCO₃ and more preferable being CaCO₃.

The hydrogenation reaction is preferably carried out at the pressure below about 2 Kg, more preferably at below about 1 Kg and still more preferably at about 0.1-1 kg.

Preferably the hydrogenation reaction is carried out at about 10-40° C., more preferably at about 15-40° C. and still more preferably at about 15-35° C.

Preferably the hydrogenation reaction is carried out in a solvent selected from alcohols, ketones, hydrocarbon solvents, ester solvents, tetrahydrofuran, water and a mixture thereof, more preferable solvent is selected from alcoholic solvent, water and a mixture thereof. Preferable alcoholic solvent is selected from methanol, ethanol, isopropyl alcohol, tert-butyl alcohol and n-butyl alcohol, more preferable alcoholic solvent is methanol or ethanol. Preferable ketonic solvent is selected from acetone, methyl isobutyl ketone and methyl ethyl ketone, more preferable ketonic solvent is acetone. Preferable hydrocarbon solvent is toluene. Preferable ester solvent is ethyl acetate.

The words “substantially pure cis” refers to cis-isomer having its trans-isomer content in about 4% or less of the contents of the cis and trans isomers put together.

Preferably “substantially pure cis” refers to cis-isomer having its trans-isomer content in less than about 2%, more preferably to cis-isomer having its trans-isomer content in less than about 1%, and still more preferably to cis-isomer having its trans-isomer content in less than about 0.5% of the contents of the cis and trans isomers put together.

The hydrogenation of 4-(3,4-dichlorophenyl)-3,4-dihydro-N-methyl-1(2H)-naphthalenimine if carried out according to prior art resulted in the formation of the mixture of cis-(±)-4-(3,4-dichlorophenyl)-1,2,3,4-tetrahydro-N-methyl-naphthalenamine and trans-(±)-4-(3,4-dichlorophenyl)-1,2,3,4-tetrahydro-N-methyl-naphthalenamine with trans-(±)-4-(3,4-dichlorophenyl)-1,2,3,4-tetrahydro-N-methyl-naphthalenamine in substantial content. In such a case, there is required to separate the undesired trans-(±)-4-(3,4-dichlorophenyl)-1,2,3,4-tetrahydro-N-methyl-naphthalenamine from the desired cis-(±)-4-(3,4-dichlorophenyl)-1,2,3,4-tetrahydro-N-methyl-naphthalenamine and then subject to resolution step to obtain sertraline or a salt thereof. The separation of trans-(±)-4-(3,4-dichlorophenyl)-1,2,3,4-tetrahydro-N-methyl-naphthalenamine from the desired cis-(±)-4-(3,4-dichlorophenyl)-1,2,3,4-tetrahydro-N-methyl-naphthalenamine requires the additional steps of a) conversion of the mixture of cis-(±)-4-(3,4-dichlorophenyl)-1,2,3,4-tetrahydro-N-methyl-naphthalenamine and trans-(±)-4-(3,4-dichlorophenyl)-1,2,3,4-tetrahydro-N-methyl-naphthalenamine isomers into their salts such as hydrochloride salts, b) fractional crystallization of the desired salt (HCl) of cis-(±)-4-(3,4-dichlorophenyl)-1,2,3,4-tetrahydro-N-methyl-naphthalenamine, and c) neutralization of the separated salt to convert to free base of cis-(±)-4-(3,4-dichlorophenyl)-1,2,3,4-tetrahydro-N-methyl-naphthalenamine. The separated cis-(±)-4-(3,4-dichlorophenyl)-1,2,3,4-tetrahydro-N-methyl-naphthalenamine is then subjected to resolution. By following the highly stereoselective hydrogenation, it is now possible, as a preferred process, to avoid the aforesaid additional steps and the hydrogenated product can directly be subjected to resolution step.

Thus, the novel process makes the process highly productive, commercially viable. Because of the highly stereoselective nature of the process of the invention, sertraline or a salt thereof is obtained in high yield in good purity.

According to the present invention hydrogenation can be carried preferably at low pressures and at ambient temperatures.

According to another aspect of the present invention, there is provided a process for preparing (1S-cis)-4-(3,4-dichlorophenyl)-1,2,3,4-tetrahydro-N-methyl-naphthalenamine of formula Ia: 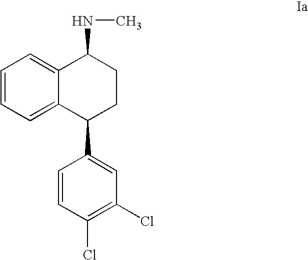
which comprises hydrogenating 4(S)-(3,4-dichlorophenyl)-3,4-dihydro-N-methyl-1(2H)-naphthalenimine of formula IIa: 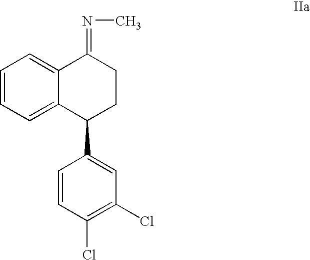
with palladium supported on an alkaline earth metal carbonate to obtain substantially pure (1S-cis)-4-(3,4-dichlorophenyl)-1,2,3,4-tetrahydro-N-methyl-naphthalenamine of formula Ia.

The preferable alkaline earth metal carbonate is CaCO₃ or BaCO₃ and more preferable being CaCO₃.

The hydrogenation reaction is preferably carried out at the pressure below about 2 Kg, more preferably at below about 1 Kg and still more preferably at about 0.1-1 kg.

Preferably the hydrogenation reaction is carried out at about 10-40° C., more preferably at about 15-40° C. and still more preferably at about 15-35° C.

Preferably the hydrogenation reaction is carried out in a solvent selected from alcohols, ketones, hydrocarbon solvents, ester solvents, tetrahydrofuran, water and a mixture thereof, more preferable solvent is selected from alcoholic solvent, water and a mixture thereof. Preferable alcoholic solvent is selected from methanol, ethanol, isopropyl alcohol, tert-butyl alcohol and n-butyl alcohol, more preferable alcoholic solvent is methanol or ethanol. Preferable ketonic solvent is selected from acetone, methyl isobutyl ketone and methyl ethyl ketone, more preferable ketonic solvent is acetone. Preferable hydrocarbon solvent is toluene. Preferable ester solvent is ethyl acetate.

The hydrogenation of 4(S)-(3,4-dichlorophenyl)-3,4-dihydro-N-methyl-1(2H)-naphthalenimine if carried out according to prior art resulted in the formation of the mixture of (1S-cis)-4-(3,4-dichlorophenyl)-1,2,3,4-tetrahydro-N-methyl-naphthalenamine and (1R-trans)-4-(3,4-dichlorophenyl)-1,2,3,4-tetrahydro-N-methyl-naphthalenamine with (1S-trans)-4-(3,4-dichlorophenyl)-1,2,3,4-tetrahydro-N-methyl-naphthalenamine in substantial content. In such a case, there is required to separate the undesired (1R-trans)-4-(3,4-dichlorophenyl)-1,2,3,4-tetrahydro-N-methyl-naphthalenamine from the desired (1S-cis)-4-(3,4-dichlorophenyl)-1,2,3,4-tetrahydro-N-methyl-naphthalenamine to obtain sertraline or a salt thereof. The separation of (1R-trans)-4-(3,4-dichlorophenyl)-1,2,3,4-tetrahydro-N-methyl-naphthalenamine from the desired (1S-cis)-4-(3,4-dichlorophenyl)-1,2,3,4-tetrahydro-N-methyl-naphthalenamine requires the additional steps of a) conversion of the mixture of (1S-cis)-4-(3,4-dichlorophenyl)-1,2,3,4-tetrahydro-N-methyl-naphthalenamine and (1R-trans)-4-(3,4-dichlorophenyl)-1,2,3,4-tetrahydro-N-methyl-naphthalenamine isomers into their salts such as hydrochloride salts, and b) fractional crystallization of the desired isomer (1S-cis)-4-(3,4-dichlorophenyl)-1,2,3,4-tetrahydro-N-methyl-naphthalenamine as salt.

By following the highly stereoselective hydrogenation, it is now possible, as a preferred process, to avoid the aforesaid additional steps.

Thus, the novel process makes the process highly productive, commercially viable. Because of the highly stereoselective nature of the process of the invention, sertraline or a salt thereof is obtained in high yield in good purity.

According to another aspect of the present invention, there is provided a process for preparing (1R-cis)-4-(3,4-dichlorophenyl)-1,2,3,4-tetrahydro-N-methyl-naphthalenamine of formula Ib: 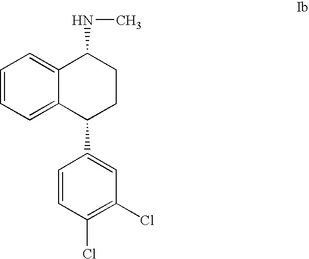
which comprises hydrogenating 4(R)-(3,4-dichlorophenyl)-3,4-dihydro-N-methyl-1(2H)-naphthalenimine of formula IIb: 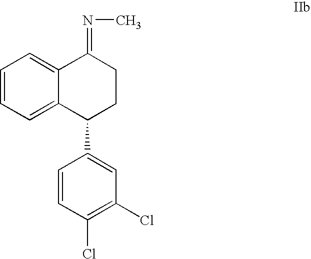
with palladium supported on an alkaline earth metal carbonate to obtain substantially pure (1R-cis)-4-(3,4-dichlorophenyl)-1,2,3,4-tetrahydro-N-methyl-naphthalenamine of formula Ib.

(1R-cis)-4-(3,4-dichlorophenyl)-1,2,3,4-tetrahydro-N-methyl-naphthalenamine obtained can be subjected to isomerisation by known methods to obtain desired isomer of sertraline.

The preferable alkaline earth metal carbonate is CaCO₃ or BaCO₃ and more preferable being CaCO₃.

The hydrogenation reaction is preferably carried out at the pressure below about 2 Kg, more preferably at below about 1 Kg and still more preferably at about 0.1-1 kg.

Preferably the hydrogenation reaction is carried out at about 10-40° C., more preferably at about 15-40° C. and still more preferably at about 15-35° C.

Preferably the hydrogenation reaction is carried out in a solvent selected from alcohols, ketones, hydrocarbon solvents, ester solvents, tetrahydrofuran, water and a mixture thereof, more preferable solvent is selected from alcoholic solvent, water and a mixture thereof. Preferable alcoholic solvent is selected from methanol, ethanol, isopropyl alcohol, tert-butyl alcohol and n-butyl alcohol, more preferable alcoholic solvent is methanol or ethanol. Preferable ketonic solvent is selected from acetone, methyl isobutyl ketone and methyl ethyl ketone, more preferable ketonic solvent is acetone. Preferable hydrocarbon solvent is toluene. Preferable ester solvent is ethyl acetate.

The invention will now be further described by the following examples, which are illustrative rather than limiting.

### EXAMPLE 1

Step-I:

The mixture of 4-(3,4-Dichlorophenyl)-3,4-dihydro-N-methyl-1(2H)-naphthalenimine (10 gm), 5% Pd/CaCO₃ (grade-21, 0.6 gm), water (2 ml) and methanol (150 ml) is taken in a hydrogenation flask and then subjected to hydrogenation under a hydrogen pressure of 0.5 Kg at 20-35° C. for 3 hours 30 minutes. The catalyst is removed by filtration and the solvent is evaporated completely under vacuum to obtain cis-(±)-4-(3,4-dichlorophenyl)-1,2,3,4-tetrahydro-N-methyl-naphthalenamine. (cis-(±):trans-(±): 99.8:0.2).

To the above reaction mass ethyl acetate (65 ml) and water (20 ml) are added and the pH is adjusted to 9.5-11.0 with aqueous sodium hydroxide (50%). The organic layer is washed with 10% sodium chloride solution (20 ml) and then subjected to carbon treatment. Then the reaction mass is heated to 45-50° C., D-(−)-mandelic acid (2.9 gm) is added at 45-50° C. and stirred for 2 hours at the same temperature. The mass is cooled to 25-35° C., stirred for 12 hours at 25-35° C., then cooled to 0-5° C. and stirred for 1 hour at the same temperature. Filtered the mass and washed with ethyl acetate, methanol (15 ml) is added and then heated to reflux. The contents are stirred for 1 hour at reflux and cooled to 25-35° C. Then the reaction mass is cooled to 0-5° C. and stirred for 1 hour at 0-5° C. Filtered the solid, washed with methanol and dried to give 4.6 gm of (1S-cis)-4-(3,4-dichlorophenyl)-1,2,3,4-tetrahydro-N-methyl-naphthalenamine mandelate (trans-(±): not detected).

Step-II:

Water (30 ml) and ethyl acetate (35 ml) are added to (1S-cis)-4-(3,4-dichlorophenyl)-1,2,3,4-tetrahydro-N-methyl-naphthalenamine mandelate (obtained in step-I), cooled to 10-18° C. and aqueous sodium hydroxide (50%) is slowly added for 1 hour 30 minutes at 10-18° C. (to adjust the pH to 9.5-11.0). The contents are stirred for 30-45 minutes, separated the aqueous layer and discarded it. Activated carbon (0.25 gm) is added to the reaction mass, stirred for 15-30 minutes, filtered and washed with ethyl acetate (5 ml). Distilled off ethyl acetate under reduced pressure until the mass temperature reaches to 50-55° C., n-butanol (30 ml) is added, stirred for 30 minutes, cooled to 5-15° C. and then conc. hydrochloric acid is slowly added during 20 minutes at 5-15° C. (to adjust the pH to below 2). The contents are stirred for 3 hours at 5-15° C., filtered the mass and washed with diisopropyl ether (5 ml). Diisopropyl ether (30 ml) is added to the solid, cooled to 5-15° C. and stirred for 1 hour at 5-15° C. Filtered the solid and dried at 45-50° C. to give 2.5 gm of sertraline hydrochloride (HPLC purity: 99.9%).

### EXAMPLE 2

The mixture of 4(S)-(3,4-Dichlorophenyl)-3,4-dihydro-N-methyl-1(2H)-naphthalenimine (10 gm), 5% Pd/CaCO₃ (grade-21, 0.6 gm), water (2 ml) and methanol (150 ml) is taken in a hydrogenation flask and then subjected to hydrogenation under a hydrogen pressure of 0.5 Kg at 20-35° C. for 3 hours 30 minutes. Then filtered the mass and washed with methanol to obtain (1S-cis)-4-(3,4-dichlorophenyl)-1,2,3,4-tetrahydro-N-methyl-naphthalenamine (1S-cis:1S-trans:99.8:0.2). The resulting mass is distilled under vacuum and then subjected to carbon treatment. Conc. hydrochloric acid is added to the reaction mass at 10° C. to adjust the pH below 2 and stirred for 2 hours at 10° C. The resulting mass is centrifuged, washed with methanol, the wet cake is slurried in methanol (40 ml) and dried to give 8 gm of (1S-cis)-4-(3,4-dichlorophenyl)-1,2,3,4-tetrahydro-N-methyl-naphthalenamine hydrochloride (1R-trans: not detected).

### EXAMPLE 3

Example 1 is repeated by using the solvent ethanol in step-I instead of methanol to give 2.4 gm of sertraline hydrochloride (HPLC purity: 99.8%).

### EXAMPLE 4

Example 1 is repeated by using the solvent acetone in step-I instead of methanol to give 2.3 gm of sertraline hydrochloride (HPLC purity: 99.7%).

### EXAMPLE 5

Step-I:

The mixture of 4-(3,4-Dichlorophenyl)-3,4-dihydro-N-methyl-1(2H)-naphthalenimine (10 gm), 5% Pd/CaCO₃ (grade-21, 0.6 gm), water (2 ml) and methanol (100 ml) is taken in a hydrogenation flask and then subjected to hydrogenation under a hydrogen pressure of 0.5 Kg at 20-35° C. for 3 hours 30 minutes. Then filtered the mass and washed with methanol to obtain cis-(±)-4-(3,4-dichlorophenyl)-1,2,3,4-tetrahydro-N-methyl-naphthalenamine (cis-(±):trans-(±):99.7:0.3). The resulting mass is distilled under vacuum and then subjected to carbon treatment. Conc. hydrochloric acid is added to the reaction mass at 10° C. to adjust the pH below 2 and stirred for 2 hours at 10° C. The resulting mass is centrifuged, washed with methanol, the wet cake is slurried in methanol (40 ml) and dried to give 8 gm of cis-(±)-4-(3,4-dichlorophenyl)-1,2,3,4-tetrahydro-N-methyl-naphthalenamine hydrochloride (trans-(±): not detected).

Step-II:

Ethyl acetate (60 ml) and water (40 ml) are added to cis-(±)-4-(3,4-dichlorophenyl)-1,2,3,4-tetrahydro-N-methyl-naphthalenamine hydrochloride (obtained in step-I) and the contents are cooled to 10-20° C. Then 50% aqueous sodium hydroxide (7.8 ml) is added at 10-20° C. during 1 hour 30 minutes (pH: 9.5-11.0), stirred for 1 hour, the aqueous layer is separated and discarded it. The ethyl acetate layer is washed with distilled water (14 ml) and discarded the aqueous layer. The contents are heated to 40-50° C., D-(−)-mandelic acid (2.8 gm) is added, stirred for 2 hours at 40-50° C. and then stirred at 25-30° C. for 12 hours. The reaction mass is cooled to 0-5° C., filtered the mass and washed with ethyl acetate (6.3 ml). Methanol (20 ml) is added to the wet cake, heated to reflux temperature and refluxed for 30-45 minutes. Then the reaction mass is cooled to 0-5° C. and stirred for 2 hours at the same temperature. The resulting solid is filtered, washed with 7 ml of methanol and dried at 60-65° C. to give 4.2 gm of (1S-cis)-4-(3,4-dichlorophenyl)-1,2,3,4-tetrahydro-N-methyl-naphthalenamine mandelate.

Step-III:

Water (25 ml) and ethyl acetate (30 ml) are added to (1S-cis)-4-(3,4-dichlorophenyl)-1,2,3,4-tetrahydro-N-methyl-naphthalenamine mandelate (obtained in step-II), cooled to 10-18° C. and 50% aqueous sodium hydroxide (2.6 ml) is slowly added for 1 hour 30 minutes at 10-18° C. (pH: 9.5-11.0). The contents are stirred for 30-45 minutes, separated the aqueous layer and discarded it. Activated carbon (0.3 gm) is added to the reaction mass, stirred for 15-30 minutes, filtered and washed with ethyl acetate (5 ml). Distilled off ethyl acetate under reduced pressure until the mass temperature reaches to 50-55° C., n-butanol (30 ml) is added, stirred for 30 minutes, cooled to 5-15° C. and then conc. hydrochloric acid (1.5 ml) is slowly added during 45 minutes at 5-15° C. (pH: 1.0). The contents are stirred for 3 hours at 5-15° C., filtered the material, washed with diisopropyl ether (40 ml) and dried at 45-50° C. to give 2.1 gm of sertraline hydrochloride (HPLC purity: 99.8%).

1. A process for the preparation of cis-(±)-4-(3,4-dichlorophenyl)-1,2,3,4-tetrahydro-N-methyl-naphthalenamine of the formula I: 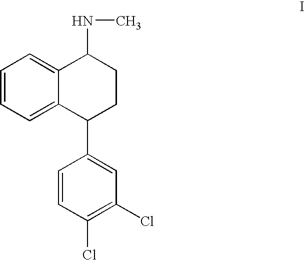
which comprises hydrogenating 4-(3,4-dichlorophenyl)-3,4-dihydro-N-methyl-1(2H)-naphthalenimine of the formula II: 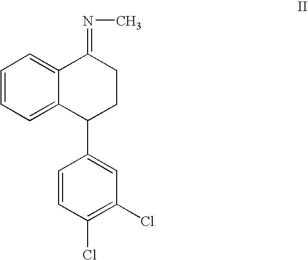
with palladium supported on an alkaline earth metal carbonate to obtain substantially pure cis-(±)-4-(3,4-dichlorophenyl)-1,2,3,4-tetrahydro-N-methyl-naphthalenamine of formula I.

2. The process as claimed in claim 1, wherein the alkaline earth metal carbonate is CaCO₃ or BaCO₃.

3. The process as claimed in claim 2, wherein the alkaline earth metal carbonate is CaCO₃.

4. The process as claimed in claim 1, wherein the hydrogenation reaction is carried out at a pressure below about 2 Kg.

5. The process as claimed in claim 4, wherein the hydrogenation reaction is carried out at a pressure below about 1 Kg.

6. The process as claimed in claim 5, wherein the hydrogenation reaction is carried out at a pressure of about 0.1-1 Kg.

7. The process as claimed in claim 1, wherein the hydrogenation reaction is carried out at a temperature of about 10-40° C.

8. The process as claimed in claim 7, wherein the hydrogenation reaction is carried out at a temperature of about 15-40° C.

9. The process as claimed in claim 8, wherein the hydrogenation reaction is carried out at a temperature of about 15-35° C.

10. The process as claimed in claim 1, wherein the hydrogenation reaction is carried out in a solvent selected from alcohols, ketones, hydrocarbon solvents, esters, tetrahydrofuran, water and mixtures thereof.

11. The process as claimed in claim 10, wherein the solvent is selected from alcoholic solvent, water and mixtures thereof.

12. The process as claimed in claim 11, wherein the alcoholic solvent is selected from the group consisting of methanol, ethanol, isopropyl alcohol, tert-butyl alcohol and n-butyl alcohol.

13. The process as claimed in claim 12, wherein the alcoholic solvent is methanol or ethanol.

14. The process as claimed in claim 10, wherein the ketonic solvent is selected from the group consisting of acetone, methyl isobutyl ketone and methyl ethyl ketone.

15. The process as claimed in claim 14, wherein the ketonic solvent is acetone.

16. The process as claimed in claim 10, wherein the hydrocarbon solvent is toluene.

17. The process as claimed in claim 10, wherein the ester solvent is ethyl acetate.

18. The process as claimed in claim 1, wherein the product obtained comprises the trans isomer in an amount of about 4% or less of the contents of the cis and trans isomers combined.

19. The process as claimed in claim 18, wherein the product obtained comprises the trans isomer in an amount of less than about 2% of the contents of the cis and trans isomers combined.

20. The process as claimed in claim 19, wherein the product obtained comprises the trans isomer in an amount of less than about 0.5% of the contents of the cis and trans isomers combined.

21. The process as claimed in claim 20, wherein the product obtained has no detectable levels of the trans isomer.

22. A process for the preparation of (1S-cis)-4-(3,4-dichlorophenyl)-1,2,3,4-tetrahydro-N-methyl-naphthalenamine of the formula Ia: 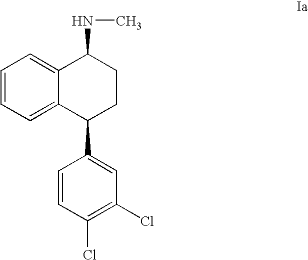
which comprises hydrogenating 4(S)-(3,4-dichlorophenyl)-3,4-dihydro-N-methyl-1(2H)-naphthalenimine of the formula IIa: 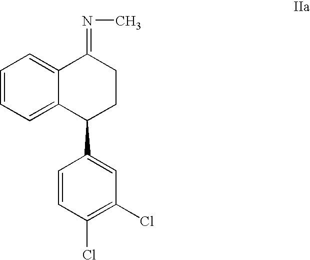
with palladium supported on an alkaline earth metal carbonate to obtain substantially pure (1S-cis)-4-(3,4-dichlorophenyl)-1,2,3,4-tetrahydro-N-methyl-naphthalenamine of the formula Ia.

23. The process as claimed in claim 22, wherein the alkaline earth metal carbonate is CaCO₃ or BaCO₃.

24. The process as claimed in claim 23, wherein the alkaline earth metal carbonate is CaCO₃.

25. The process as claimed in claim 22, wherein the hydrogenation reaction is carried out at a pressure below about 2 Kg.

26. The process as claimed in claim 25, wherein the hydrogenation reaction is carried out at a pressure below about 1 Kg.

27. The process as claimed in claim 26, wherein the hydrogenation reaction is carried out at a pressure about 0.1-1 Kg.

28. The process as claimed in claim 22, wherein the hydrogenation reaction is carried out at a temperature of about 10-40° C.

29. The process as claimed in claim 28, wherein the hydrogenation reaction is carried out at a temperature of about 15-40° C.

30. The process as claimed in claim 29, wherein the hydrogenation reaction is carried out at a temperature of about 15-35° C.

31. The process as claimed in claim 22, wherein the hydrogenation reaction is carried out in a solvent selected from alcohols, ketones, hydrocarbon solvents, esters, tetrahydrofuran, water and mixtures thereof.

32. The process as claimed in claim 31, wherein the solvent is selected from alcoholic solvent, water and mixtures thereof.

33. The process as claimed in claim 32, wherein the alcoholic solvent is selected from methanol, ethanol, isopropyl alcohol, tert-butyl alcohol and n-butyl alcohol.

34. The process as claimed in claim 33, wherein the alcoholic solvent is methanol or ethanol.

35. The process as claimed in claim 31, wherein the ketonic solvent is selected from acetone, methyl isobutyl ketone and methyl ethyl ketone.

36. The process as claimed in claim 35, wherein the ketonic solvent is acetone.

37. The process as claimed in claim 31, wherein the hydrocarbon solvent is toluene.

38. The process as claimed in claim 31, wherein the ester solvent is ethyl acetate.

39. The process as claimed in claim 22, wherein the product obtained has 1R-trans isomer in an amount of about 4% or less of the contents of the 1S-cis and 1R-trans isomers combined.

40. The process as claimed in claim 39, wherein the product obtained has 1R-trans isomer in an amount of less than about 2% of the contents of the 1S-cis and 1R-trans isomers combined.

41. The process as claimed in claim 40, wherein the product obtained has 1R-trans isomer in an amount of less than about 0.5% of the contents of the 1S-cis and 1R-trans isomers combined.

42. The process as claimed in claim 41, wherein the product obtained has no detectable amounts of the 1R-trans isomer.

43. A process for the preparation of (1R-cis)-4-(3,4-dichlorophenyl)-1,2,3,4-tetrahydro-N-methyl-naphthalenamine of the formula Ib: 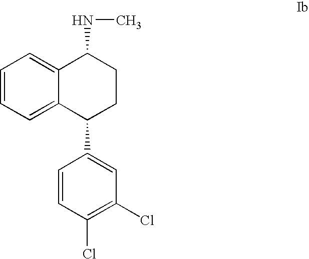
which comprises hydrogenating 4(R)-(3,4-dichlorophenyl)-3,4-dihydro-N-methyl-1(2H)-naphthalenimine of the formula IIb: 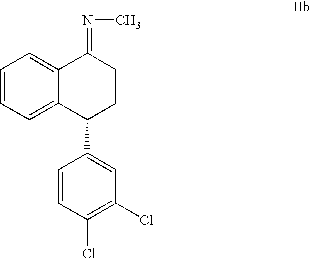
with palladium supported on an alkaline earth metal carbonate to obtain substantially pure (1R-cis)-4-(3,4-dichlorophenyl)-1,2,3,4-tetrahydro-N-methyl-naphthalenamine of the formula Ib.

44. The process as claimed in claim 43, wherein the alkaline earth metal carbonate is CaCO₃ or BaCO₃.

45. The process as claimed in claim 44, wherein the alkaline earth metal carbonate is CaCO₃.

46. The process as claimed in claim 43, wherein the hydrogenation reaction is carried out at a pressure below about 2 Kg.

47. The process as claimed in claim 46, wherein the hydrogenation reaction is carried out at a pressure below about 1 Kg.

48. The process as claimed in claim 47, wherein the hydrogenation reaction is carried out at a pressure of about 0.1-1 Kg.

49. The process as claimed in claim 43, wherein the hydrogenation reaction is carried out at a temperature of about 10-40° C.

50. The process as claimed in claim 49, wherein the hydrogenation reaction is carried out at a temperature of about 15-40° C.

51. The process as claimed in claim 50, wherein the hydrogenation reaction is carried out at a temperature of about 15-35° C.

52. The process as claimed in claim 43, wherein the hydrogenation reaction is carried out in a solvent selected from alcohols, ketones, hydrocarbon solvents, esters, tetrahydrofuran, water and mixtures thereof.

53. The process as claimed in claim 52, wherein the solvent is selected from an alcoholic solvent, water and mixtures thereof.

54. The process as claimed in claim 53, wherein the alcoholic solvent is selected from methanol, ethanol, isopropyl alcohol, tert-butyl alcohol and n-butyl alcohol.

55. The process as claimed in claim 54, wherein the alcoholic solvent is methanol or ethanol.

56. The process as claimed in claim 52, wherein the ketonic solvent is selected from acetone, methyl isobutyl ketone and methyl ethyl ketone.

57. The process as claimed in claim 56, wherein the ketonic solvent is acetone.

58. The process as claimed in claim 52, wherein the hydrocarbon solvent is toluene.

59. The process as claimed in claim 52, wherein the ester solvent is ethyl acetate.

60. The process as claimed in claim 1, further comprising step (c) resolving the cis-(±)-4-(3,4-dichlorophenyl)-1,2,3,4-tetrahydro-N-methyl-naphthalenamine formed in step (b) to obtain (1S-cis)-4-(3,4-dichlorophenyl)-1,2,3,4-tetrahydro-N-methyl-naphthalenamine and optionally converting it to a pharmaceutically acceptable salt by a known method.

61. The process as claimed in claim 60, wherein resolving agent used in the resolution is D-(−)-mandelic acid and the pharmaceutically acceptable salt is sertraline hydrochloride.

62. The process as claimed in claim 1, further comprising step (c) crystallizing cis-(±)-4-(3,4-dichlorophenyl)-1,2,3,4-tetrahydro-N-methyl-naphthalenamine from the reaction mass obtained in step(b) as hydrochloride salt, converting the salt into cis-(±)-4-(3,4-dichlorophenyl)-1,2,3,4-tetrahydro-N-methyl-naphthalenamine free base using a base, resolving cis-(±)-4-(3,4-dichlorophenyl)-1,2,3,4-tetrahydro-N-methyl-naphthalenamine to obtain (1S-cis)-4-(3,4-dichlorophenyl)-1,2,3,4-tetrahydro-N-methyl-naphthalenamine and optionally converting it to a pharmaceutically acceptable salt.

63. The process as claimed in claim 62, wherein the base used to convert cis-(±)-4-(3,4-dichlorophenyl)-1,2,3,4-tetrahydro-N-methyl-naphthalenamine hydrochloride salt to cis-(±)-4-(3,4-dichlorophenyl)-1,2,3,4-tetrahydro-N-methyl-naphthalenamine free base is sodium hydroxide, the resolution agent used in resolution step is D-(−)-mandelic acid and the pharmaceutically acceptable salt is sertraline hydrochloride.

64. The process as claimed in claim 60, wherein the hydrogenation reaction is carried out in n-butyl alcohol.

65. The process as claimed in claim 64, wherein hydrochloric acid is added to obtain sertraline hydrochloride.

66. The process as claimed in claim 63, wherein the hydrogenation reaction is carried out in n-butyl alcohol.

67. The process as claimed in claim 66, wherein hydrochloric acid is added to obtain sertraline hydrochloride.

68. The process as claimed in claim 33, wherein the alcoholic solvent is n-butyl alcohol.

69. The process as claimed in claim 68, wherein hydrochloric acid is added to obtain (1S-cis)-4-(3,4-dichlorophenyl)-1,2,3,4-tetrahydro-N-methyl-naphthalenamine.

70. The process as claimed in claim 12, wherein the alcoholic solvent is n-butyl alcohol.

71. The process as claimed in claim 70, wherein hydrochloric acid is added to obtain cis-(±)-4-(3,4-dichlorophenyl)-1,2,3,4-tetrahydro-N-methyl-naphthalenamine.

72. The process as claimed in claim 54, wherein the alcoholic solvent is n-butyl alcohol.

73. The process as claimed in claim 72, wherein hydrochloric acid is added to obtain (1R-cis)-4-(3,4-dichlorophenyl)-1,2,3,4-tetrahydro-N-methyl-naphthalenamine.
